(19) United States
(12) Patent Application Publication
(10) Pub. No.: US 2009/0259134 A1
(43) Pub. Date: Oct. 15, 2009

(54) Symptom recording patient interface system for a portable heart monitor

(76) Inventor: Glenn N. Levine, Pearland, TX (US)

Correspondence Address: LOUIS VENTRE, JR, 2483 OAKTON HILLS DRIVE, OAKTON, VA, 22124-1530, US

(21) Appl. No.: 12/101,989

(22) Filed: Apr. 12, 2008

(51) Int. Cl. A61B 5/0428

(52) U.S. Cl. 600/523

(57) ABSTRACT

A patient interface device contemporaneously records patient symptoms with an automated recording of heart rhythm data from a portable heart monitor. The patient interface device includes the portable heart monitor and a computer with a user input means to enter symptoms and store them in computer memory. Code means operable on the computer enables entry and recording of the symptoms. Where the portable heart monitor is activated automatically, a signaling means alerts the patient that it has begun recording and that symptoms experienced by the patient should be entered and recorded. Preferably, the electronic record of patient symptoms and the recording of the patient's abnormal heart rhythm event are linked such that they are accessed and used together.

### FIELD OF INVENTION

In the field of diagnostic testing involving the medical evaluation of a heart condition of a living body, a device is disclosed for contemporaneous recording of patient symptoms with an automated recording of heart rhythm data from a portable heart monitor.

### BACKGROUND OF THE INVENTION

The term “heart rhythm” as used herein is intended to be broadly construed to include any measurement relating to a heartbeat, including for example, a abnormal heart rhythm (including various slow and fast heart rhythm), and electrocardiogram (ECG) abnormality (including an ST segment change, QT segment duration, and others).

Many patients will report symptoms to their physician such as “dizziness,” “fast heart beating,” “skipped beats,” “lightheadedness,” “shortness of breath” or “chest pain.” While the cause of such symptoms can in many cases be identified by simple measures such as the history, physical exam, and ECG, frequently a clear cause for such symptoms cannot be identified.

In many of these patients, it is suspected that they might have had arrhythmias, or abnormal beating of the heart. Both too slow a heartbeat and too fast a heartbeat can cause such symptoms. If the patient is having such symptoms while the patient is hooked up to an ECG machine, it can easily be determined if the patient is having an arrhythmia that is causing the symptoms. However, because the patient may experience such symptoms only once every several days, once a week, or even once a month, most of the time one cannot record the heart rhythm during the symptoms.

To solve this problem, portable cardiac event monitors are used. The phrases “portable cardiac event monitor,” “event monitor,” “cardiac monitor,” and “a portable monitor of heart rhythms” are used interchangeably herein and are intended to include any of the portable devices used to monitor heart rhythms, including for example, portable Holter monitors, and “hybrids” such as event monitors combined with Holter monitors.

An event monitor is a small (approximately 2″×2″×½″) electronic device that consists of the event monitor box along with several ECG electrodes that are attached to the skin. The event monitor is worn continuously by the patient, usually for approximately 1 month.

When the patient experiences some form of symptom, the patient pushes a button on the event monitor, and the event monitor records the heart rhythm. The event monitor stores the recorded heart rhythm when the “record” button is pushed by the patient, and can then later transmit the stored data, usually over a telephone system, to a central office. Typically, event monitors will store and record the heart rhythm that occurred in the 30-60 seconds immediately prior to the patient pushing the record button, and for 30-60 seconds immediately after the patient pushed the record button.

The problem with current event monitors is that they lack information on the patient symptoms. What is critical for the physician to know is whether a patient's symptoms actually correlate with an abnormal heart rhythm. In other words, if a patient complains of episodes of lightheadedness, but it can be demonstrated that the recorded heart rhythm during these periods is completely normal, then the physician can be confident that the patient's lightheadedness is not caused by an arrhythmia. If on the other hand every time the patient experiences lightheadedness, the heart rhythm is either too slow or too fast, then the physician knows that this particular symptom is caused by too slow or too fast a heart rate.

No currently available event monitor records what symptom the patient has when he or she pushes the record button, or records if the patient is having any symptoms if the event monitor itself automatically detects an arrhythmia and records (stores) the arrhythmia.

Current event monitors contain only one button that the patient can push to record the heart rhythm—the patient cannot input any other information regarding what symptom or symptoms they are experiencing. Thus, patients are sometimes given a “log book” to write down their symptoms. Unfortunately, patients frequently do not carry this log book around with them, and frequently fail to record any symptoms they had. In addition, the time that the patient records in the log book as having had the symptom usually does not correlate well with the time that the event monitor recorded the heart rhythm. Thus, it is difficult if not impossible to try to correlate the patient's symptoms with recorded heart rhythms.

Some companies that provide event monitors to patients will call the patient at a certain time and ask the patient if he or she has had any symptoms during the preceding days or weeks. Unfortunately, patients usually do not remember all their symptoms and the exact time that they had them, so again one is not able to correlate a patient's symptoms and the recorded heart rhythms.

Finally, as noted above, at present, all available event monitors have only a single button for the patient to push when they have a symptom. While one knows that the patient experienced some symptom, one cannot know what that symptom was. It could have been a symptom such as chest pain, feeling the heart beating fast, the heart skipping beats, chest pains, shortness of breath, or even briefly fainting. Without knowing the exact symptom the patient was experiencing, it is all but futile to try to figure out if the patient's specific complaint that brought him or her to the physician's office correlates with an important arrhythmia that requires treatment.

The system becomes even more muddled since third party vendors are frequently the ones who send physicians the list of recorded heart rhythms. These companies simply cannot provide an accurate report of what symptoms, if any, the patient was having when the heart rhythms were recorded. It has been reported that at least one third party vendor simply discards the diary of symptoms that patients are supposed to keep.

One more layer of complexity is added by automated heart rhythm monitors. All event monitors have the one-button configuration that allows a patient to press the button if they are experiencing a symptom. Some more advanced event monitors will not only record the heart rhythm when the patient hits the record button, but they also have software programs that trigger the event monitor to automatically record and store certain arrhythmias, even if the patient did not press the record button. When these additional recordings are downloaded to the physician, it becomes even more difficult for the physician to figure out if the patient was having any symptoms when these arrhythmias were recorded.

At present, some event monitors will “beep” (or alert the patient in some similar manner) when they automatically record an arrhythmia. This alerts the patient that the event monitor has recorded the heart rhythm, but does not allow the patient to respond in any way. When the recorded abnormal rhythms are sent to the physician who has ordered the test (either via hard copy or electronically), the physician is faced with even more recorded heart rhythms to examine, but even less understanding of whether the heart rhythms recorded by the device were associated with any symptoms, and if they were associated with a symptom, the physician cannot determine what symptom that was.

A “Holter Monitor” adds the ability to take a continuous recording of heart activity and typically has no patient activation button. A Holter monitor is worn by the patient and it continuously electronically records the patient's heart rhythm. While an event monitors is usually worn for approximately 4 weeks and only record approximately 1 minute of the heart rhythm at a time (when the patient pushes a “record” button or when the device automatically detects an abnormal heart rhythm), a Holter monitor is usually worn for 1-2 days and continuously records and stores the heart rhythm.

A Holter monitor is usually prescribed to the patient when they have frequent symptoms (e.g. multiple times each day). They also classically do not have any way for the patient to directly record and input into the device if they are having any symptoms. Although the patient may sometimes be given a “log book” or “diary” to record their symptoms, the patients frequently forget to record in the log book their symptoms, or there is a poor correlation between the time they actually write in their log book when they had symptoms, and what is later found on the Holter monitor recordings of abnormal heart rhythms.

Thus, similar to event monitors, Holter monitors suffer from the same deficiency: it is usually impossible for physicians to try to figure out if the patient was having any symptoms when the recorded abnormal heart rhythms occurred.

“Hybrid” cardiac monitors are also known to integrate hybrid technology and features of event monitors and Holter monitors. They may continuously record the heart rhythm for longer periods of time (e.g. weeks), may also automatically detect, record, and store abnormal heart rhythms, may automatically send an alert to the company that owns or leases out the monitors, may have “record” buttons for the patient to push if they experience a symptom, and various combinations and permutations of the above.

Some cardiac monitors also have software and functionality that allows the monitor to recognize any changes in the ST segment of the ECG. The ST segment of the ECG is a part of the ECG that is associated with myocardial ischemia and/or myocardial infarction. The ST segment may become “depressed” from the ECG baseline if the heart experiences myocardial ischemia (not enough blood carrying oxygen) or may become “elevated” from the ECG baseline if a coronary artery becomes completely blocked and the heart begins to have a myocardial infarction. Some cardiac monitors can also detect and analyze other changes to the ECG, including changes in what is called the QRS complex, QT interval, and other parameters. Some such cardiac monitors only analyze and record characteristics or aspects of the ECG itself, while others incorporate such technology of ECG analysis into a “hybrid monitor.”

The term “hybrid monitor”, as used in the patent application, encompasses any device that incorporates combinations of the features of classic event monitors and Holter monitors, as well as devices that can detect ST segment changes and other features of the QRS complex, QT interval, or other ECG parameters, with any or several of the basic goals of recording the heart rhythm, detecting abnormal heart rhythms, alerting the patient of an abnormal heart rhythm, alerting the device owner or leasing company of an abnormal heart rhythm, quantifying abnormal heart rhythms, detecting ST segment changes, alerting the patient of detected ST segment changes, alerting the device owner or leaser of ST segment changes, or any other analysis of the electrocardiogram (such as QRS complex, QT interval, etc.) or heart rhythm. These hybrid cardiac monitors may or may not have a “record” button, for the patient to push if he/she experiences any symptoms.

None of existing hybrid cardiac monitors allows the patient to input and record the symptom or symptoms they are experiencing, or to record if they are experiencing any symptoms at the time that the device alerts the patient (through a beep, vibration, light signal, or other mechanism) that it has detected an abnormal heart rhythm event. As used herein when the context so permits, the phrase “patient's abnormal heart rhythm event,” or “abnormal heart rhythm event,” or the term “event,” includes any event recorded or analyzed by a heart monitor, such as a change in the ST segment or any other analysis of the electrocardiogram (such as QRS complex, QT interval, etc.) or heart rhythm.

Portable cardiac monitors may also be categorized as pre-symptom and post symptom monitors. The pre symptom monitor is typically a looping memory event monitor, Holter monitor, or a “hybrid” monitor that includes electrodes attachable to a person's chest. The electrodes capture ECG rhythms before the cardiac event monitor is triggered and this is why they are referred to as pre-symptom monitors. A pre-symptom monitor is sometimes necessary when there is a potential for a patient to lose consciousness upon the occurrence of an arrhythmia.

The post-symptom monitor does not have chest electrodes. One type of post-symptom monitor is worn on the wrist. When symptoms occur, the patient presses a button to trigger a heart rhythm recording. A second type of post-symptom monitor is a device that the patient carries. When symptoms occur, the patient presses the electrodes on the device against their chest and presses a button to trigger the ECG recording.

The present invention provides a solution that allows the patient to input and record what symptoms, if any, they experienced when a heart rhythm is recorded by a heart monitor. For example, the symptom may be recorded when the patient pushes the “record” button (in the case of event monitors and hybrid cardiac monitors) or while simply wearing a Holter monitor or hybrid cardiac monitor.

An embodiment of the present invention includes a signaling means to notify the patient that it has detected an abnormal heart rhythm event by beeping, flashing a light, vibrating, or other means, and to contemporaneously prompt the patient or a companion to input into the device what symptoms, if any the patient was experiencing at the time of the event.

An embodiment of the present invention electronically links the recorded heart rhythm (or ST segment change) with the inputted symptom or symptoms. This allows, at a later time when the information is transmitted or downloaded, the physician or device owner/leaser to have an accurate, exact, print out of what the heart rhythm was when the patient experienced a symptom, and what symptoms the patient experienced when the device automatically detected an abnormal heart rhythm event.

### BRIEF SUMMARY OF THE INVENTION

A patient interface device integrating a portable monitor of heart rhythms and a computer comprising a user input means, memory, and code means to enable contemporaneous creation of an electronic record of patient symptoms experienced with a recording of a patient's abnormal heart rhythm event. The user input means may comprise a keypad or touch screen that preferably enables a patient or nearby companion to make, amend and record selections of preprogrammed symptoms. The code means preferably enables alteration of the pre-programmed selections, for example, commensurate with a doctor's orders. Alternatively, the user input means is a contemporaneous voice message file that is temporally associated with the recording of the patient's abnormal heart rhythm event. Preferably, the electronic record of patient symptoms and patient's abnormal heart rhythm event recording are linked, such that they are accessed and used together and such linkage may include a temporal indication on both the electronic record and the recording. For automatically activated monitors, the device may include a signaling means, such as a vibrator, a flashing light, and an audio signal generator, and code means to activate the signaling means to alert the patient that it has detected an abnormal heart rhythm event and prompt a user to input into the device any symptoms the patient experienced.

### BRIEF DESCRIPTION OF THE DRAWINGS

FIG. 1 is a front view of an embodiment of the patient interface device that includes a pre symptom event monitor.

FIG. 2 is a diagram of the patient interface device component interrelationships.

### DETAILED DESCRIPTION

In the following description, reference is made to the accompanying drawings, which form a part hereof and which illustrate several embodiments of the present invention. The drawings and the preferred embodiments of the invention are presented with the understanding that the present invention is susceptible of embodiments in many different forms and, therefore, other embodiments may be utilized and structural and operational changes may be made without departing from the scope of the present invention.

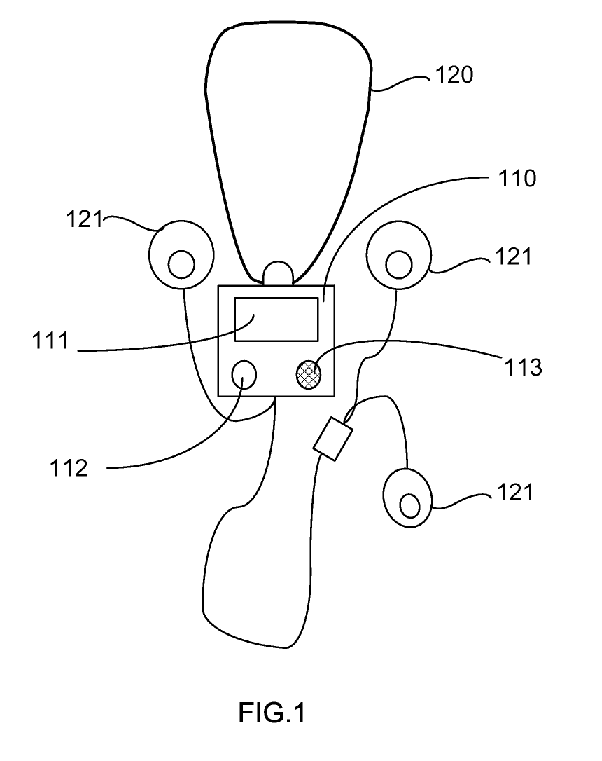

FIG. 1 shows an embodiment of the patient interface device that includes a portable monitor of heart rhythms. This portable monitor is in the form of a pre symptom event monitor, which has electrodes (121) that are attachable to a patient's chest. A lanyard (120) is typically secured around a patient's neck, over the shoulder (like a handbag), or on the arm (like a walkman or I-tunes). The lanyard (120) preferably holds a combined recording system and computer (110). The recording system is capable of storing several minutes of the individual's ECG record. A start button (112) enables patient activation of the recording system when the patient experiences a symptomatic period of arrhythmia.

The computer includes a user input means, which in this embodiment is a select button (113) and LCD screen (111). The computer further includes computer memory and code means that is operable on the computer to enable a user to employ the select button (113) make selections of applicable pre-programmed patient experiences. The user is a patient, or a companion, making the selections and thus creating and storing in the computer memory an electronic record of patient symptoms experienced contemporaneously with a recording of the patient's abnormal heart rhythm event by the portable monitor of heart rhythms.

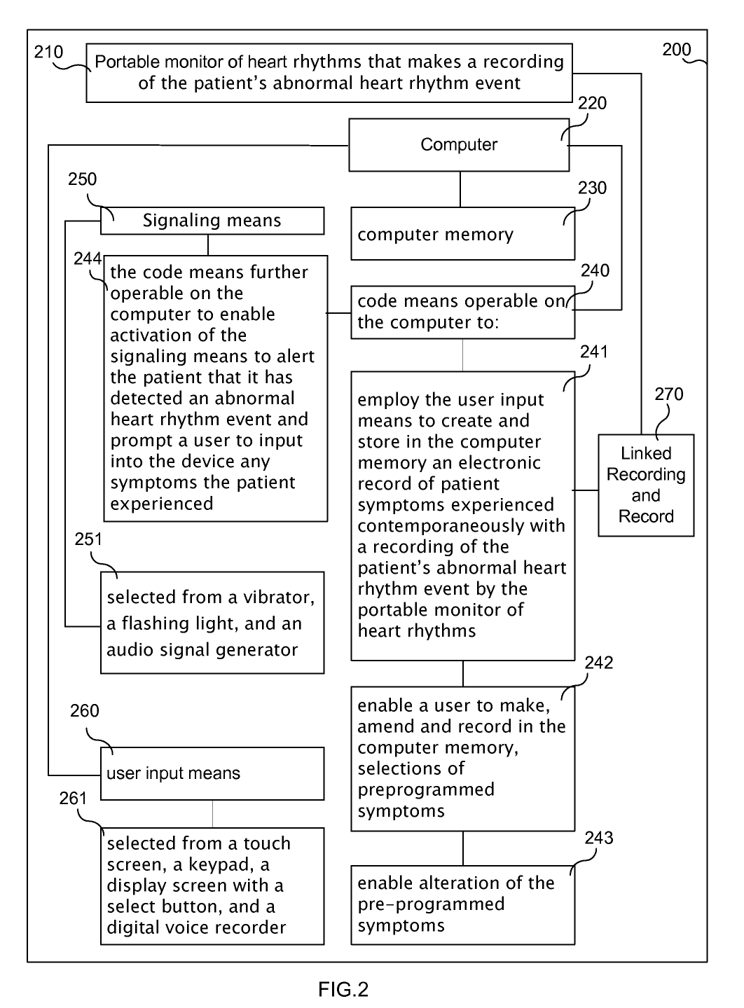

FIG. 2 is a representation of various preferred embodiments of the patient interface device and its component interrelationships. The patient interface device (200) includes a portable monitor of heart rhythms that makes a recording of the patient's abnormal heart rhythm event (210). Devices of this kind are well known in the art.

The patient interface device (200) also includes a computer (220) comprising a user input means (260); computer memory (230); and, code means operable on the computer (240). In some embodiments where the portable monitor of heart rhythms is automatically activated to begin recording, the patient interface device (200) includes a signaling means (250) to alert the patient that it has begun recording and for the patient or a companion to enter and record any symptoms experienced by the patient. Preferably, the electronic record of patient symptoms and the recording of the patient's abnormal heart rhythm event are linked (270) such that they are accessed and used together and such linkage may include a temporal indication on both the electronic record and the recording.

The user input means (260) may be any typical computer user input means. Examples (261) include a touch screen that may be activated by a users finger to make selections of pre-programmed symptoms, or to type in the symptoms; a keypad preferably in combination with a display screen to enable a user to type in the symptoms, a display screen with a select button (113) to enable selection of pre-programmed symptoms; and a digital voice recorder that creates an electronic voice recording of oral statements about the patient's symptoms. LCD and plasma display screens are well known in the art.

Examples of preferable pre-programmed symptoms are chest pain, short of breath, dizziness, lightheaded, fainted, fast heartbeats, skipped heartbeats.

While one select button (113) is shown, several select and scroll buttons may be used to allow a user to scroll to pre-programmed symptoms that they are experiencing, to select/deselect symptoms, and to record the selections. A button may also have multiple functions, for example, a record button may also serve as a start button for patients to push when they experience a symptom.

The computer memory (230) is readable and writable to enable storage of the symptoms and for other functions enabled by the code means operable on the computer (240).

The code means operable on the computer (240) enables a user to:
(241)—employ the user input means (260) to create and store in the computer memory an electronic record of patient symptoms experienced contemporaneously with a recording of the patient's abnormal heart rhythm event by the portable monitor of heart rhythms;
optionally: (242)—make, amend and record in the computer memory, selections of preprogrammed symptoms
optionally: (243)—enable alteration of the pre-programmed symptoms; and,
optionally, when a signaling means (250) is present: (244)—enable activation of the signaling means (250) to alert the patient that it has detected an abnormal heart rhythm event and prompt a user to input into the device any symptoms the patient experienced.

The signaling means (250) may be any means to alert the patient or companion to enter any symptoms experienced by the patient associated with the automated start of recording of the patient abnormal heart rhythm event. Examples include a vibrator such as one similar to that commonly found on cellular telephones that quietly reminds the patient to enter symptoms; a flashing light such as a small red LED that would be an obvious indication; and, an audio signal generator such as one similar to a cellular telephone ring.

An alternative embodiment of the patient interface device (200) is also illustrated in FIG. 2. For this embodiment, the patient interface device (200) is an instrument carried by the patient and it works in conjunction with a separate and electronically-connected event monitor. Essentially, the patient interface device (200) has all of the components shown in FIG. 2. However, the portable monitor of heart rhythms that makes a recording of the patient's abnormal heart rhythm event (210) has a connection with, but is not within the same unit as, the patient interface device (200).

For this embodiment, the patient interface device first includes a computer that has a connection with a portable monitor of heart rhythms. Preferably, the computer is a hand-held device similar in size to cell phone. The connection may be hard wired or preferably wireless.

This patient interface device further includes a user input means (260) connected to the computer. This is of the type previously described above.

This patient interface device further includes computer memory (230) connected to the computer, which, as described above, is also readable and writable.

This patient interface device (200) further includes code means operable on the computer (240) to enable a user to employ the user input means to create and store in the computer memory an electronic record of patient symptoms experienced contemporaneously with a recording of the patient's abnormal heart rhythm event by the portable monitor of heart rhythms (241). The electronic record of patient symptoms is linked (270) to the recording of patient's abnormal heart rhythm event through the connection with the portable monitor of heart rhythms. Preferably, the linkage enables the electronic record and the recording to be accessed and used together and may have a temporal indication on both the electronic record and the recording.

The first 5 following examples apply primarily to pre-symptom monitors, those monitors worn with leads attached to the chest. Example 6 applies to post-symptom monitors, those without continuously worn chest leads.

### EXAMPLE 1

When the Symptom Reporting Patient Interface System is built in to a conventional event monitor, the device functions in the following manner when the patient experiences a symptom:

The patient experiences a symptom (such as lightheadedness or fast heart beat).

The patient pushes the “symptom/record” button, and the system records the heart rhythm for 30-60 seconds immediately prior to the “symptom/record” button being pushed and for 30-60 seconds immediately after the “symptom/record” button being pushed

The Symptom Reporting Patient Interactive System then prompts the patient (with a text message on the patient interface screen and a “beeping sound”, vibration, or other method to notify the patient to respond) to input what symptom or symptoms they just experienced.

The patient interface screen displays a list of programmable symptoms (e.g. “chest pain”, “lightheadedness”, “shortness of breath”) for the patient to chose from and displays a message “Please Select Your Symptom or Symptoms” (or a similar message).

The patient uses a scroll button or buttons (which can be variably configured) to highlight the symptom that he/she experienced.

The patient pushes the “select” button to check off that symptom.

If the patient has experienced more than one symptom, he/she then highlights and selects any additional symptom. If he/she accidentally selects the wrong symptom, then he/she can push the “select” button (actually a select/deselect button) one more time to “un-check” that symptom.

After one or more symptom has been checked off (selected) by the patient, the Symptom Reporting Patient Interface System will prompt the patient to push again the “record” button when he/she is done selecting all the symptoms he/she has experienced. To do this, it will display a message on the screen such as “please push the record button when you are finished selecting symptoms.” The patient then pushes the “record” button again to input and record the symptoms that he/she has experienced. This records the symptoms and couples the selected symptoms with the heart rhythm that was just recorded and stored by the Symptom Reporting Patient Interface System.

At a given time, the data stored in the Symptom Reporting Patient Interface System is downloaded to a computer that can print out (hard copy or electronically) the recorded heart rhythms and systems. The print out shows each specific recorded symptom (or symptoms) and the exact temporal corresponding heart rhythm. In this manner, the physician can easily and reliably know what the heart rhythm was each time the patient experienced a symptom and activated the Symptom Reporting Patient Interface System.

If the system is coupled with a cell phone-like device, the symptom and recorded heart rhythm are instantaneously and automatically “called in” and downloaded to a central control station, where the recorded heart rhythm and symptom(s) is immediately available to the physician or device leaser.

The device not only is programmed with the above patient prompt messages, but is programmed to “trouble shoot” if the patient becomes confused and hits the wrong button at the wrong time, or does not remember what do to next, and display other helpful and prompting messages.

### EXAMPLE 2

When the Symptom Reporting Patient Interface System is built in to a conventional event monitor that is programmed to additionally detect and record on its own any pre-programmed abnormal heart rhythm, the device will function in the following manner when it automatically detects an abnormal heart rhythm. These steps build on those steps described in detail above:

The device automatically detects an abnormal heart rhythm. The device has been pre-programmed as to the criteria for considering a heart rhythm to be abnormal (such as slower or faster than a certain number of beats per minute).

When the device detects an abnormal heart rhythm, it “beeps,” vibrates or uses other similar manners to alert the patient that is has automatically detected an abnormal heart rhythm. These alerts may become louder or more intense if the patient does not respond to the alert within a pre-programmed period of time. This alert initiates the patient's input of symptoms in to the Symptom Reporting Patient Interface System.

The patient interface screen instructs the patient to select any symptom or symptoms that they were experiencing, similar to described above. The patient can also select the choice of “no symptom” to input that they were not experiencing any symptom at the time the device detected an abnormal heart rhythm. This is clinically extremely important, as physicians will often treat (such as with a pacemaker) an abnormal heart rhythm that causes symptoms (such as lightheadedness), but feel that it is best not to treat certain abnormal heart rhythms if they do not cause the patient any symptoms.

Once the patient has selected which symptoms, if any, the patient has experienced, the patient pushes the “record” button, to record these inputted symptoms (or “no symptoms”), electronically coupling the detected abnormal heart rhythm with the symptoms, if any, that the patient experienced when the abnormal heart rhythm was detected.

At a given time, the data stored in the Symptom Reporting Patient Interface System is downloaded to a computer that can print out (hard copy or electronically) the recorded heart rhythms and systems. The print out shows the recorded symptoms for each abnormal heart rhythm immediately above the displayed heart rhythm. In this manner, the physician can easily and reliably know what symptom the patient was having for each abnormal heart rhythm that was automatically detected and recorded.

If the system is coupled with a cell phone-like device, the symptom and recorded heart rhythm can be instantaneously and automatically “called in” and downloaded to a central control station, where the recorded heart rhythm and symptom(s) is immediately available to the physician or device leaser.

The device not only is programmed with the above patient prompt messages, but is programmed to “trouble shoot” if the patient becomes confused and hits the wrong button at the wrong time, or does not remember what do to next, and display other helpful and prompting messages.

### EXAMPLE 3

When the Symptom Reporting Patient Interface System is built in to a conventional Holter monitor (which just continuously records the heart rhythm over a period of several days), the device will function in the following manner when the patient experiences a symptom. Note that the steps described below are similar for the most part to those described above:

The patient experiences a symptom (such as lightheadedness or fast heart beat)

The patient pushes the “symptom/record” button. This activates the Symptom Reporting Patient Interactive System to begin the symptom input process. Since a conventional Holter monitor continuously records the heart rhythm, it is already recording the heart rhythm.

The Symptom Reporting Patient Interactive System then prompts the patient (with a text message on the patient interface screen and a “beeping sound”, vibration, or other method to remind the patient to respond) to input what symptom or symptoms they just experienced.

The patient interface screen displays a list of programmable symptoms (e.g. “chest pain”, “lightheadedness”, “shortness of breath”) for the patient to chose from and displays a message “Please Select Your Symptom or Symptoms” (or a similar message).

The patient uses the scroll buttons (which can be variably configured) to highlight the symptom that he/she experienced.

The patient pushes the “select” button to check off that symptom

If the patient has experienced more than one symptom, he/she then highlights and selects any additional symptom. If he/she accidentally selects the wrong symptom, then he/she can push the “select” button (actually a select/deselect button) one more time to “un-check” that symptom.

After one or more symptom has been checked off (selected) by the patient, the Symptom Reporting Patient Interface System will prompt the patient to push again the “record” button when he/she is done selecting all the symptoms he/she has experienced. To do this, it will display a message on the screen such as “please push the record button when you are finished selecting symptoms”. The patient then pushes the “record” button again to input and record the symptoms that he/she has experienced. This records the symptoms and couples the selected symptoms with the heart rhythm that was recorded and stored by the Symptom Reporting Patient Interface System at the time that the patient pushed the “symptom/record” button

At a given time, the data stored in the Symptom Reporting Patient Interface System is downloaded to a computer that can print out (hard copy or electronically) the recorded heart rhythms and systems. The print out shows each specific recorded symptom (or symptoms) and the exact temporal corresponding heart rhythm. In this manner, the physician can easily and reliably know what the heart rhythm was each time the patient experienced a symptom and activated the Symptom Reporting Patient Interface System.

If the system is coupled with a cell phone-like device, the symptom and recorded heart rhythm can be instantaneously and automatically “called in” and downloaded to a central control station, where the recorded heart rhythm and symptom(s) is immediately available to the physician or device leaser.

The device not only is programmed with the above patient prompt messages, but is programmed to “trouble shoot” if the patient becomes confused and hits the wrong button at the wrong time, or does not remember what do to next, and display other helpful and prompting messages.

### EXAMPLE 4

When the Symptom Reporting Patient Interface System is built in to a hybrid cardiac monitor that is programmed to additionally detect and record on its own any pre-programmed abnormal heart rhythm, and/or to detect any ST segment changes, the device will function in the following manner when the patient experiences a symptom:

The patient experiences a symptom (such as lightheadedness or fast heart beat).

The patient pushes the “symptom/record” button, and the system records the heart rhythm for 30-60 seconds immediately prior to the “symptom/record” button being pushed and for 30-60 seconds immediately after the “symptom/record” button being pushed (if it is not programmed to simply continuously record the heart rhythm, as in a “Holter monitor mode”, in which case it will have already recorded and stored the heart rhythm). In hybrid monitors that are programmed to detect ST segment changes, the device also records the ST segment during this period, and whether it has detected any ST segment changes (or other changes to the electrocardiogram).

The Symptom Reporting Patient Interactive System then prompts the patient (with a text message on the patient interface screen and a “beeping sound,” vibration, or other method to notify the patient to respond) to input what symptom or symptoms they just experienced.

The patient interface screen displays a list of programmable symptoms (e.g. “chest pain”, “lightheadedness”, “shortness of breath”) for the patient to chose from and displays a message “Please Select Your Symptom or Symptoms” (or a similar message).

The patient uses the scroll buttons (which can be variably configured) to highlight the symptom that he/she experienced.

The patient pushes the “select” button to check off that symptom

If the patient has experienced more than one symptom, he/she then highlights and selects any additional symptom. If he/she accidentally selects the wrong symptom, then he/she can push the “select” button (actually a select/deselect button) one more time to “un-check” that symptom.

After one or more symptom has been checked off (selected) by the patient, the Symptom Reporting Patient Interface System will prompt the patient to push again the “symptom/record” button when he/she is done selecting all the symptoms he/she has experienced. To do this, it will display a message on the screen such as “please push the record button when you are finished selecting symptoms”. The patient then pushes the “symptom/record” button again to input and record the symptoms that the patient has experienced. This records the symptoms and couples the selected symptoms with the heart rhythm that was just recorded and stored by the Symptom Reporting Patient Interface System

At a given time, the data stored in the Symptom Reporting Patient Interface System is downloaded to a computer that can print out (hard copy or electronically) the recorded heart rhythms and systems. The print out shows each specific recorded symptom (or symptoms) and the exact temporal corresponding heart rhythm (and or ST changes or other measured electrocardiogram parameters). In this manner, the physician can easily and reliably know what the heart rhythm was each time the patient experienced a symptom and activated the Symptom Reporting Patient Interface System. Likewise, the physician can easily and reliably know what symptoms, if any, the patient was experiencing each time the device detected (and recorded) an abnormal heart rhythm, ST segment change, or other programmed electrocardiogram parameter.

If the system is coupled with a cell phone-like device, the symptom and recorded heart rhythm (and ST changes) can be instantaneously and automatically “called in” and downloaded to a central control station, where the recorded heart rhythm and symptom(s) is immediately available to the physician or device leaser.

The device not only is programmed with the above patient prompt messages, but is programmed to “trouble shoot” if the patient becomes confused and hits the wrong button at the wrong time, or does not remember what do to next, and display other helpful and prompting message.

### EXAMPLE 5

When the Symptom Reporting Patient Interface System is built in to a hybrid cardiac monitor that is programmed to additionally detect and record on its own any pre-programmed abnormal heart rhythm, the device will function in the following manner when it detects an abnormal heart rhythm and/or any ST segment changes or other ECG abnormalities:

The device automatically detects an abnormal heart rhythm. The device has been pre-programmed as to the criteria for considering a heart rhythm to be abnormal (such as slower or faster than a certain number of beats per minute) and/or pre-programmed as to the criteria for considering ST segment changes or other ECG manifestations as abnormal.

When the device detects an abnormal heart rhythm, it “beeps,” vibrates or uses other similar manners to alert the patient that is has automatically detected an abnormal heart rhythm. These alerts may become louder or more intense if the patient does not respond to the alert within a pre-programmed period of time. This alert initiates the patient's input of symptoms in to the Symptom Reporting Patient Interface System.

The patient interface screen instructs the patient to select any symptom or symptoms that they were experiencing, similar to described above. The patient can also select the choice of “no symptom” to input that they were not experiencing any symptom at the time the device detected an abnormal heart rhythm. This is clinically extremely important, as physicians will often treat (such as with a pacemaker) an abnormal heart rhythm that causes symptoms (such as lightheadedness), but feel that it is best not to treat certain abnormal heart rhythms if they do not cause the patient any symptoms. Similarly, it is important to know if a patient does or does not experience any symptom (such as chest pain) when an ST segment change or other ECG abnormality is detected.

Once the patient has selected which symptoms, if any, the patient has experienced, the patient pushes the “record” button, to record these inputted symptoms (or “no symptoms”), electronically coupling the detected abnormal heart rhythm with the symptoms, if any, that the patient experienced when the abnormal heart rhythm was detected.

At a given time, the data stored in the Symptom Reporting Patient Interface System is downloaded to a computer that can print out (hard copy or electronically) the recorded heart rhythms and systems. The print out shows the recorded symptoms for each abnormal heart rhythm immediately above the displayed heart rhythm. In this manner, the physician can easily and reliably know what symptom the patient was having for each abnormal heart rhythm that was automatically detected and recorded.

If the system is coupled with a cell phone-like device, the symptom and recorded heart rhythm can be instantaneously and automatically “called in” and downloaded to a central control station, where the recorded heart rhythm and symptom(s) is immediately available to the physician or device leaser.

The device not only is programmed with the above patient prompt messages, but is programmed to “trouble shoot” if the patient becomes confused and hits the wrong button at the wrong time, or does not remember what do to next, and display other helpful and prompting messages.

### EXAMPLE 6

When the Symptom Reporting Patient Interface System is built in to a post-symptom monitor, such as a device that applied to the chest when the patient experiences a symptom, the device functions in the following manner when the patient experiences a symptom:

The patient experiences a symptom (such as lightheadedness or fast heart beat).

The patient pushes the “symptom/record” button, in the case where the device is worn on the wrist, or pushes a button and applies the device to the chest, in the case of devices that are applied to the chest when the patient experiences a symptom, and the system records the heart rhythm.

The Symptom Reporting Patient Interactive System then prompts the patient (with a text message on the patient interface screen and a “beeping sound”, vibration, or other method to notify the patient to respond) to input what symptom or symptoms they just experienced.

The patient interface screen displays a list of programmable symptoms (e.g. “chest pain”, “lightheadedness”, “shortness of breath”) for the patient to chose from and displays a message “Please Select Your Symptom or Symptoms” (or a similar message).

The patient uses a scroll button or buttons (which can be variably configured) to highlight the symptom that he/she experienced.

The patient pushes the “select” button to check off that symptom.

If the patient has experienced more than one symptom, he/she then highlights and selects any additional symptom. If he/she accidentally selects the wrong symptom, then he/she can push the “select” button (actually a select/deselect button) one more time to “un-check” that symptom.

After one or more symptom has been checked off (selected) by the patient, the Symptom Reporting Patient Interface System will prompt the patient to push again the “record” button when he/she is done selecting all the symptoms he/she has experienced. To do this, it will display a message on the screen such as “please push the record button when you are finished selecting symptoms.” The patient then pushes the “record” button again to input and record the symptoms that he/she has experienced. This records the symptoms and couples the selected symptoms with the heart rhythm that was just recorded and stored by the Symptom Reporting Patient Interface System.

At a given time, the data stored in the Symptom Reporting Patient Interface System is downloaded to a computer that can print out (hard copy or electronically) the recorded heart rhythms and systems. The print out shows each specific recorded symptom (or symptoms) and the exact temporal corresponding heart rhythm. In this manner, the physician can easily and reliably know what the heart rhythm was each time the patient experienced a symptom and activated the Symptom Reporting Patient Interface System.

If the system is coupled with a cell phone-like device, the symptom and recorded heart rhythm are instantaneously and automatically “called in” and downloaded to a central control station, where the recorded heart rhythm and symptom(s) is immediately available to the physician or device leaser.

The device not only is programmed with the above patient prompt messages, but is programmed to “trouble shoot” if the patient becomes confused and hits the wrong button at the wrong time, or does not remember what do to next, and display other helpful and prompting messages.

The above-described embodiments including the drawings are examples of the invention and merely provide illustrations of the invention. Other embodiments will be obvious to those skilled in the art. Thus, the scope of the invention is determined by the appended claims and their legal equivalents rather than by the examples given.

What is claimed is:

1. A patient interface device comprising:
(a) a portable monitor of heart rhythms that makes a recording of the patient's abnormal heart rhythm event; and,
(b) a computer comprising a user input means; computer memory; and, code means operable on the computer to enable a user to employ the user input means to create and store in the computer memory an electronic record of patient symptoms experienced contemporaneously with a recording of the patient's abnormal heart rhythm event by the portable monitor of heart rhythms.

2. The patient interface device of claim 1 wherein the user input means is selected from a group consisting of a touch screen, a keypad, a display screen with a select button, and a digital voice recorder.

3. The patient interface device of claim 2 wherein the user input means is functional with the code means to enable a user to make, amend and record in the computer memory, selections of preprogrammed symptoms.

4. The patient interface device of claim 2 wherein the code means enables alteration of the pre-programmed symptoms.

5. The patient interface device of claim 1 further comprising a signaling means and wherein the code means is further operable on the computer to enable activation of the signaling means to alert the patient that it has detected an abnormal heart rhythm event and prompt a user to input into the device any symptoms the patient experienced.

6. The patient interface device of claim 5 wherein the signaling means is selected from a group consisting of a vibrator, a flashing light, and an audio signal generator.

7. The patient interface device of claim 1 wherein the electronic record of patient symptoms and the recording of the patient's abnormal heart rhythm event are linked such that they are accessed and used together.

8. A patient interface device comprising:
(a) a computer that has a connection with a portable monitor of heart rhythms;
(b) a user input means connected to the computer;
(c) computer memory connected to the computer; and,
(d) code means operable on the computer to enable a user to employ the user input means to create and store in the computer memory an electronic record of patient symptoms experienced contemporaneously with a recording of the patient's abnormal heart rhythm event by the portable monitor of heart rhythms, wherein such electronic record of patient symptoms is linked to the recording of patient's abnormal heart rhythm event through the connection with the portable monitor of heart rhythms.
